(19) United States
(12) Patent Application Publication
(10) Pub. No.: US 2008/0262080 A1
(43) Pub. Date: Oct. 23, 2008

(54) Allergy Suppressive Agent

(75) Inventor: Hirofumi Tachibana, Fukuoka (JP)

Correspondence Address: BIRCH STEWART KOLASCH & BIRCH, PO BOX 747, FALLS CHURCH, VA, 22040-0747, US

(73) Assignee: KYUSHU UNIVERSITY, NATIONAL UNIVERSITY CORPORATION, Fukuoka-shi (JP)

(21) Appl. No.: 11/664,622

(22) PCT Filed: Oct. 5, 2005
(86) PCT No.: PCT/JP2005/018451

§ 371 Date: Apr. 4, 2008

(30) Foreign Application Priority Data
Oct. 5, 2004 (JP) 2004-292500

(51) Int. Cl. A61K 31/352; C07D 311/36; A61P 37/08

(52) U.S. Cl. 514/456; 549/403

(57) ABSTRACT

The present invention provides a novel agent/composition using a bioregulatory function of a food ingredient.

Namely, it is intended to provide an allergy suppressive agent comprising chrysin, more specifically speaking, a histamine release inhibitory agent comprising chrysin; a composition comprising chrysin that is a composition for treating a disease or condition relating to the production of IgE; a composition comprising chrysin that is a composition for treating a disease or condition relating to a high-affinity IgE receptor; and a composition comprising chrysin that is a composition for treating a disease or condition relating to the overproduction of IL-4. It is preferable that the allergy suppressive agent and histamine release inhibitory agent according to the present invention further comprise apigenin.

### TECHNICAL FIELD

The present invention relates to an allergy suppressive agent. The agent according to the present invention can be in the form of a food, a drink or a medicine.

### BACKGROUND ART

There are a large number of allergy-mediated diseases. Major examples thereof include pollinosis, allergic rhinitis, atopic dermatitis, bronchial asthma, urticaria, collagen disease, pneumonia hypersensitivity, and immune deficiency and so on. It is said that one in three people in Japan is affected by allergic symptoms. Therefore, there is an urgent need to develop an effective treatment for allergy-mediated diseases.

In recent years, a number of food products and the like are being developed, which comprise an active ingredient obtained from a natural source that is efficacious in alleviating allergic symptoms, for example, yogurt using a specific lactic acid bacterium, a drink comprising mint polyphenol comprised in peppermint, and health food tablets and drinks comprising polyphenol and naringenin chalcone comprised in tomato skin. These active ingredients obtained from natural sources have attracted public attention, are highly acceptable for daily consumption because they are extracted from natural sources and are safer compared with synthetic chemicals; moreover, they not only alleviate unpleasant symptoms but also ameliorate allergic diathesis per se.

Antiallergic effects of flavonoids and polyphenols obtained from natural sources have been reported in several documents.

Patent Document 1 discloses a polyphenol mixture obtained from an unripe fruit harvested in the fruit thinning season. In this fruit polyphenol mixture, simple polyphenol compounds such as a coffeic acid derivative, a p-coumaric acid derivative, flavan-3-ols (catechins), flavonols (quercetin glycosides), dihydrochalcones (phloretin glycosides) and so on and high-molecular polyphenol compounds such as condensed tannins amount to the major part of the composition thereof. Accordingly, it is reported that this polyphenol mixture is efficacious as an antioxidant, a hypotensive agent, an antimutagenic agent, an allergic inhibitor, a cariostatic agent and a deodorant.

Patent Document 2 discloses a hyarulonidase activity inhibitory agent which comprises at least one substance selected from among theaflavin monogallate A, theaflavin monogallate B and theaflavin digallate as the active ingredient. Since hyarulonidase is causative of inflammation and its activity is inhibited by an antiinflammatory agent or an antiallergic agent, it is reported that these active ingredients inhibit hyarulonidase activity and thus alleviate various inflammations and allergies. Similarly, relating to compounds obtained from tea leaves, Patent Document 3 discloses an antiallergic agent comprising, as the active ingredient(s), 3-O-methylgalloylepigallocatechin and/or 4-O-methylgalloylepigallocatechin that are separated and collected from a polyphenol fraction extracted from tea leaves with an aqueous solvent. It is reported that these catechins suppress type I and type IV allergic reactions. Patent Document 4 discloses an antiallergic agent characterized by comprising, as the active ingredient, at least one kind of polyphenol selected from strictinin and methylated derivatives thereof that can be separated and collected from a polyphenol fraction extracted from tea leaves with an aqueous solvent. These active ingredients were found via a screening using an effect of suppressing IgE production as an indication.

Moreover, Patent Document 5 discloses an antiallergic agent and an antiinflammatory agent comprising a phloglucinol derivative which is a component of a Mallotus japonicus pericarp extract. It is reported that this active ingredient has effects of suppressing histamine release and suppressing prostaglandin E2 production.

Based on a finding that an extract of a plant of the family Labiatae specifically suppresses TNF production, Patent Document 6 discloses an antiallergic cosmetic composition characterized by comprising a component obtained by extracting a plant of the family Labiatae with an organic solvent. It is reported that this composition has an immunological function-suppressing action, an antiallergic effect on type I disease models and an effect of ameliorating atopic dermatitis. Similarly, relating to a substance obtained from a plant of the family Labiatae, Patent Document 7 discloses an antiallergic substance obtained from a hot water extract of Perilla frutescens crispa which is characterized by being a brown substance having a mast cell degranulation-inhibitory activity, comprising saccharides and amino acids as constituents thereof, having a molecular weight of 13,500 and having a specific rotation [α]_{D} (0.25, H₂O) of −0.4. Furthermore, Patent Document 8 discloses a histamine release suppressive agent which is obtained from an alcoholic extract of perilla seeds and comprises one or more kinds of compounds selected from apigenin, chrysoeriol, luteolin and rosmarinic acid. It is reported that this histamine release suppressive agent can make the release of histamine from sensitized cells difficult in the course of the onset of type I allergy so as to enable efficacious treatment and prevention of an allergic disease.

Apigenin and luteolin are found in other plants. Patent Document 9 discloses an antiallergic composition comprising, as the active ingredient, an Impatiens textorii petal extract comprising at least one kind of compound selected from the group consisting of luteorin, apigenin and apigenin-7-O-glucoside. It is reported that this composition has effects of alleviating inflammations in atopic dermatitis, pollinosis, food allergy, urticaria and so on and relieving intense itching in allergic skin diseases. Since the active ingredient shows a remarkable suppressive effect on itching caused by a histamine release agent in this case, it is considered that one of the mechanisms of the anti-itch effect of the active ingredient resides in the suppression of degranulation.

On the other hand, flavonoids comprised in honeybee and propolis have been reported in several documents. It is reported that honeybee comprises p-hydroxybenzoic acid, p-coumaric acid, cis- and trans-abscisic acids, cinnamic acid, pinobanksin, pinocembrin, chrysin, etc. and has an antioxidant effect and so on (see, for example, Non-Patent Documents 1 to 4). It is also known that chrysin has an effect of suppressing the action of aromatase that converts androstenedione and testosterone into estrogen. Thus, chrysin has been marketed as a supplement for improving hormone balance.

However, research relating to honeybee and allergies have been mainly focused on allergy to honeybee. As a study aiming at alleviating allergic symptoms, there can be enumerated nothing but one reporting that the topical administration of a mixture of honeybee with beeswax and olive oil is efficacious against atopic dermatitis and vulgar psoriasis (Non-Patent Document 5) and it has never been reported that a specific component of honeybee exerts an antiallergic effect.
Patent document 1: Japanese Patent Laid-Open No. H07-285876 (Japanese Patent No. 3521155) or Japanese Patent Laid-Open No. 2002-47196
Patent document 2: Japanese Patent No. 3242997
Patent document 3: Japanese Patent Laid-Open No. 2000-159670
Patent document 4: Japanese Patent Laid-Open No. 2002-12545
Patent document 5: Japanese Patent Laid-Open No. 2003-73265
Patent document 6: Japanese Patent Laid-Open No. H06-293652
Patent document 7: Japanese Patent Laid-Open No. H09-20672 (Japanese Patent No. 3071669)
Patent document 8: Japanese Patent Laid-Open No. 2000-86510
Patent document 9: Japanese Patent Laid-Open No. 2001-278796
Non-patent Document 1: Nele Gheldof et al.: J. Agric. Food Chem. 2002, 50, 5870-5877
Non-patent Document 2: Jed W. Fahey et al.: J. Agric. Food Chem. 2002, 50, 7472-7476
Non-patent Document 3: Lihu Yao et al.: J. Agric. Food Chem. 2004, 52, 210-214
Non-patent Document 4: Jed W. Fahey et al.: J. Agric. Food Chem. 2004, 52, 7472-7476
Non-patent Document 5: Al-Waili N S.: Complement Ther Med. 2003 December; 11 (4); 226-34

### SUMMARY OF THE INVENTION

The present inventor has conducted studies on the bioregulatory functions of food ingredients. Based on a finding that a honeybee has an antiallergic effect, the inventor further conducted detailed studies and consequently identified chrysin as the active ingredient of an allergy suppressive agent, thereby completing the present invention.

### BRIEF DESCRIPTION OF THE DRAWINGS

FIG. 1 shows the suppressive effect of chrysin on germline IgE heavy chain transcription (εGT). IL-4 (25 U/ml) and chrysin at various concentrations were added to human mature B cell line DND39 cells and then the cells were incubated for 48 hours. mRNA was collected from the cells and cDNA was synthesized. An RT-PCR amplification product was electrophoresed on an agarose gel and transferred onto a membrane and εGT was thus detected. GAPDH (glyceraldehyde-3-phosphate dehydrogenase) was employed as a control. For each concentration or the control, the blot in the right side indicates the result in a 1/10 amount.

FIG. 2 shows the suppressive effect of chrysin on the phosphorylation of STAT6. DND39 cells were treated with IL-4 (100 U/ml) and chrysin at various concentrations and then lysed. Each cell lysate was subjected to immunoprecipitation using anti-STAT6 antibody. The immunoprecipitate was subjected to 8% SDS-PAGE and transferred onto a nitrocellulose membrane and phosphorylated STAT6 was detected.

FIG. 3 shows lowering in high-affinity IgE receptor (FcεRI) expression caused by chrysin. By using antihuman FcRI antibody (CRA1), the FcεRI expression amount on the surface of human basophil-like cell line KU812 cells was analyzed by flow cytometry. Cells having been incubated by adding DMSO alone were compared with cells having been incubated by adding chrysin. The ratio of overexpression of FcεRI is indicated in percentage.

FIG. 4 shows the suppressive effect of chrysin on the expression of the proteins constituting the high-affinity IgE receptor (FcεRI). The expression of each of the FcεRI α- and γ-chains was detected by collecting cells having been incubated by adding chrysin, lysing the cells, conducting immunoprecipitation, subjecting the immunoprecipitate to DS-PAGE and then blotting. β-Actin was employed as a control.

FIG. 5 shows the suppressive effect of chrysin on mRNA transcription of the proteins constituting the high-affinity IgE receptor (FcεRI). The mRNA expression of each of the FcεRI α- and γ-chains was detected by RT-PCR and southern hybridization using specific probes. G3PDH (glyceraldehyde-3-phosphate dehydrogenase) was employed as a control.

FIG. 6 shows the suppressive effect of orally administered chrysin on STAT6 phosphorylation. Spleen lymphocytes collected from mice, to which chrysin had been orally administered continuously, were stimulated with LPS or LPS+IL-4 (500 U/ml) and phosphorylated STAT6 was detected by immunoprecipitation using anti-STAT6 antibody and immunoblotting.

FIG. 7 shows the effect of chrysin on mRNA expression levels of the FcεRI-constituting chains of human peripheral blood basophils. Peripheral blood basophils (1×10⁶ cells/mL) collected from the vein of a donor were incubated in a medium comprising 25 M of chrysin or dimethyl sulfoxide (DMSO) for 24 hours. Then, RNA was extracted and the mRNA expression level of each of the FcεRI α- and γ-chains was examined by the RT-PCR method.

FIG. 8 shows the effect of chrysin on histamine release from human basophils. Human basophil cell line KU812 cells (5×10⁶ cells/mL) were incubated in Tyrode buffer comprising 1 mM of CaCl₂, to which 25 μM of chrysin had been added, and then stimulated with A23187. Histamine released from the cells and histamine cumulated in the cells were quantified to thereby determine the histamine release rate. Comparison was made with a control system free from chrysin.

### DISCLOSURE OF THE INVENTION

The present invention provides an allergy suppressive agent, in particular a type I allergy suppressive agent, comprising chrysin.

Chrysin (5,7-dihydroxyflavone) has the following structure.

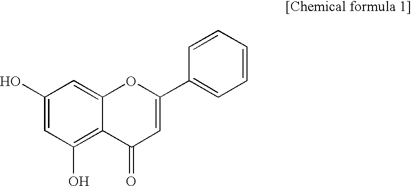

Chrysin to be used in the present invention can be prepared from a naturally occurring substance comprising chrysin such as honeybee, carrot seed or a tree of a certain kind.

Except in a special case, the term “allergy” is used herein in a meaning commonly employed in the field of the present invention. Except in a special case, therefore, the term “allergy” as used herein can be defined as a systemic or topical damage to the living body based on an immune reaction and includes allergies based on humoral immune reactions (i.e., allergies of types I, II and III) and allergies based on cellular immunity mediated by sensitized lymphocytes (i.e., allergies of type IV). Examples of “allergy” or “allergic disease” include atopic dermatitis, pollinosis, allergic rhinitis, allergic conjunctivitis, bronchial asthma, urticaria, collagen disease, pneumonia hypersensitivity, anaphylaxis, food allergy, chemical allergy, dust mite allergy, metal allergy and animal allergy.

Now, the onset mechanism of an allergic disease will be illustrated. A humoral immune reaction proceeds in the following order: (1) contact of an allergen with immunocompetent cells (antigen presenting cells); (2) production of an antibody owing to the synergistic effects of the antigen presenting cells, T cells and B cells mediated by a cytokine, etc.; (3) activation of effector cells by the antigen-antibody reaction; (4) release of a chemical mediator (histamine, etc.), a cytokine, etc. from the effector cells; and (5) occurrence of an allergic reaction in an organ. A cellular immune reaction proceeds in the following manner: (1) sensitization of T cells with an antigen; (2) binding of the sensitized T cells to the antigen; (3) production of a cytokine or a chemokine by the sensitized T cells, activation of other cells therewith and production of an additional cytokine; and (4) occurrence of an allergic reaction in an organ. Any allergy suppressive agent wherein chrysin is used in suppressing allergy falls within the category of the allergy suppressive agent according to the present invention, regardless of which stage it regulates.

According to detailed studies conducted by the present inventor, it has been clarified that chrysin can regulate early stages in an allergic reaction based on a humoral allergic reaction. More specifically speaking, chrysin regulates at least the IL-4-induced germline IgE heavy chain transcription stage, the STAT6 phosphorylation stage, the stage of high affinity IgE receptor expression on cell surface, the stage of expression of α- or γ-chain constituting high-affinity IgE receptor and the high-affinity IgE receptor transcription stage. Therefore, the allergy suppressive agent according to the present invention can be effectively used against allergies based on humoral immune reactions (i.e., allergies of types I, II and III), in particular, type I allergies. Chrysin, which is usable as the active ingredient of a histamine release inhibitory agent, is also effectively usable in treating various diseases or conditions on which regulation of any of the above-described stages is effective.

Accordingly, the present invention provides the following matters:

1) a histamine release inhibitory agent comprising chrysin;

2) a composition comprising chrysin for treating a disease or condition relating to the production of IgE (more specifically, for treating a disease or condition relating to IL-4-induced germline IgE heavy chain transcription and for treating a disease or condition relating to STAT6 phosphorylation);

3) a composition comprising chrysin for treating a disease or condition relating to a high-affinity IgE receptor (more specifically, for treating a disease or condition relating to the high affinity IgE receptor expression on cell surface, for treating a disease or condition relating to α- or γ-chain constituting the high-affinity IgE receptor and for treating a disease or condition relating to the high-affinity IgE receptor transcription). and

4) a composition comprising chrysin for treating a disease or condition relating to the overproduction of IL-4.

The expressions “suppression (to suppress)” an allergy and “treatment (to treat)” a disease or condition as used herein relating to the agent or composition according to the present invention include preventing or curing the allergy or the disease or conditions (hereinafter referred to simply as “allergy and the like”), regulating allergy and the like to a moderate level or arresting the progress of allergy and the like. The terms “suppression” and “treatment” involve a symptomatic therapy aiming at relieving symptoms and a fundamental therapy aiming at relieving hypersensitivity or improving allergic diathesis. These terms further involve an immediate therapy and a long-lasting prevention and/or therapy. Furthermore, they involve the prevention of new correlated symptoms following allergy and the like.

To suppress or treat allergy and the like, the agent or composition according to the invention may be used in an appropriate manner depending on the subject, disease conditions, purpose and so on. Since chrysin has a suppressive effect on IgE production, it is expected that the agent or composition according to the invention would exert a preventive effect and a long-lasting therapeutic effect on allergy and the like. In the case of using for these purposes, it seems preferable to continuously use the agent or composition according to the invention before the onset of allergy and the like or when the allergy is in an early stage and still mild.

Since IgE has an action of enhancing and sustaining the expression of a high-affinity IgE receptor, a lowering in IgE level contributes to the suppression of the high-affinity IgE receptor expression level. Owing to this suppressive effect on high-affinity IgE receptor expression, it is expected that the agent or composition according to the invention would exert an immediate effect of suppressing allergy as well as a long-lasting effect of preventing allergy. In the case of aiming at achieving an immediate effect of suppressing allergy, the agent or composition according to the invention can be used at the onset of allergy and the like or thereafter. In this case, the agent or composition according to the invention can exert an effect of preventing the subsequent onset of allergy and the like. In the case of aiming at achieving the long-lasting prevention effect, the agent or composition according to the invention can be continuously used before the onset of allergy and the like or in the mild stage. Owing to the continuous use, the high-affinity IgE receptor expression in a subject can be continuously regulated to a low level and, therefore, it is expected that the allergen sensitivity of the subject can be lowered.

The term “continuous” as used herein relating to the usage of the agent or composition according to the invention means using the agent or composition twice or more in a definite period of time (for example, several days, several weeks, several months or several years). That is, it involves a case of using in successive days as well as a case of using intermittently at definite or indefinite intervals. According to the inventor's studies, chrysin can exert its suppressive effect on the IL-4-induced STAT6 phosphorylation even when intermittently used in a low dose (see Example 3).

The “agent” or “composition” as mentioned herein may be in the form of a medicine. As discussed above, chrysin can exert its inhibitory effect on the IL-4-induced STAT6 phosphorylation even when intermittently used in a low dose and, therefore, the agent or composition according to the invention can be relatively freely taken. Thus, the agent or composition according to the invention can be in a form other than that of a medicine (for example, a food, a drink, etc.)

On the agent or composition according to the invention, it is possible to indicate the intended purposes (for example, preventing, alleviating hypersensitivity to an allergen, improving allergic diathesis, conducting a long-lasting therapy, etc., or inhibiting histamine release, suppressing IgE production, suppressing IL-4-induced germline IgE heavy chain transcription, suppressing STAT6 phosphorylation, suppressing high-affinity IgE receptor expression, suppressing high-affinity IgE receptor expression on cell surface, suppressing expression of α- or γ-chain constituting a high-affinity IgE receptor, suppressing high-affinity IgE receptor transcription, etc.) and/or practical methods of using the same (for example, a dose, dosing frequency, instructions of continuous use, a dosing period, etc.).

In the case where the agent or composition according to the invention is a medicine, the content of chrysin employed as the active ingredient can be appropriately determined depending on the purpose, conditions, age and body weight of a subject and so on. For example, a preparation can be made so as to administer from about 0.001 to about 1000 mg/kg, preferably from about 0.01 to 100 mg/kg, of chrysin once or several times per day.

According to the inventor's studies, it is clarified that chrysin is efficacious as an antiallergic agent when orally dosed in a dose of 1.68 g/day (in the case of male adults having an average body weight of 60 kg) or more. From this viewpoint, it is recommended that the agent or composition according to the invention is an agent or a composition whereby about 1.68 g/day or more of chrysin can be orally administered or dosed. Specifically, it is possible to prepare an agent or a composition which comprises 1.68 g or more of chrysin in the dose of the daily oral administration or dosing. In other words, it is possible to prepare an agent or a composition which comprises 28 mg/kg or more of chrysin in the dose of the daily oral administration or dosing. In the case of an antiallergic preparation for oral use to be administered thrice a day, more specifically speaking, the content of chrysin in a single dose is controlled to 560 mg or more. An antiallergic food (or drink) with recommendation of taking about three portions per day can comprise 560 mg or more of chrysin.

Also, the administration route and dosage form can be appropriately designed. For example, a preparation for systemic administration or a preparation for topical administration may be produced. Examples of the dosage form include an internal medicine, an external medicine, a solid, a liquid, a powder, granules, a capsule, a tablet, an ointment, a plaster, a patch, a lotion, a liniment and a suppository. Moreover, the agent or composition according to the invention may be in the form of a sustained-release preparation or a controlled-release preparation. These preparations can be produced by a method commonly employed in the art and use can be made of various pharmaceutically acceptable additives such as a filler, a binder, a disintegrating agent, a lubricating agent, a coating, a suspending agent, an emulsifier, a stabilizer, a preservative and a buffer.

Also, the agent or composition according to the invention may be in the form of a medical device or a quasi drug. It is also possible to add the agent or composition according to the invention to an ointment, a cosmetic lotion, a lotion, soap, a shampoo, a wet tissue paper and so on.

The agent or composition according to the invention may be in the form of a cosmetic, a food or a drink. The content of chrysin that is the active ingredient may be appropriately determined in accordance with the case of a medicine. Foods or drinks comprising the allergy suppressive agent according to the present invention may be a food with nutrient function claims, a food for specified health use, a health food, a nutritional supplement, an instant food, a frozen food, a health drink and so on. These products may be encapsulated, made into tablets or added to sweets, noodles, soups, seasonings (for example, mayonnaise, soybean paste (miso), soy sauce, dressing and sauce), other fermented foods, canned foods, processed meat products (for example, ham and sausage), processed milk products (for example, yogurt), pickles, soft drinks, carbonated drinks, fruit juice drinks, milk drinks, lactic acid drinks, sport drinks and so on.

The present invention further provides the following matters:

5) use of chrysin in the manufacture of an allergy suppressive agent, a histamine release inhibitory agent, a composition for treating a disease or condition relating to the production of IgE, a composition for treating a disease or condition relating to a high-affinity IgE receptor or a composition for treating a disease or condition relating to the overproduction of IL-4; and

6) a method for treating a disease or condition relating to allergy, a disease or condition relating to histamine release, a disease or condition relating to the production of IgE, a disease or condition relating to a high-affinity IgE receptor or a disease or condition relating to the overproduction of IL-4, which comprises administering a therapeutically effective amount of chrysin to a mammal with a need for such a treatment.

In a preferred mode for carrying out the present invention, chrysin is used together with apigenin. That is, the present invention provides a composition for medicines or foods which comprise chrysin and apigenin.

Apigenin has the following structure.

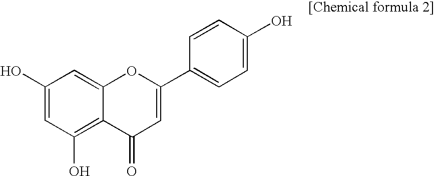

According to the inventor's detailed studies, chrysin employed in a certain amount did not suppress the expression of FcεRI in KU812 cells but potentiated the suppressive effect of apigenin on FcεRI expression. When chrysin and apigenin were used at a molar ratio of 1:5, a synergistic suppressive effect was observed on FcεRI expression (see Example 8).

Although the invention using chrysin and apigenin as the active ingredients has been illustrated above, the present invention is likely applicable to derivatives thereof (for example, methylated compounds, glycosides, glucuronic acid conjugations and sulfuric acid conjugations). Therefore, it is to be understood that use of such a derivative for the same purpose as in the present invention falls within the scope of the present invention too.

### EXAMPLES

### Example 1

### Suppression of IgE Production

### Suppression of Germline IgE Heavy Chain Transcription (εGT) and Suppression of STAT6 Phosphorylation)

### 1. Object

Although immune reactions protect the human body from foreign invaders' attacks by immune reactions, the immune reactions sometimes injure the human body. These injuries based on the immune functions are called allergies. In recent years, there has been a remarkable tendency toward an increase in the number of patients suffering from allergic reactions and growing severity of allergic diseases. It is said that one in three people in Japan are affected by have been bothered by some allergy induced diseases such as pollinosis, allergic rhinitis, atopic dermatitis and so on. Immune reactions occurring in vivo are classified into humoral immunity in which an antibody participates and cellular immunity in which no antibody participates. Allergic reactions of the types I to III belong to the former category, while type IV allergic reactions belong to the latter. Environmental allergies such as pollen allergy are caused by the type I allergy, while it is suspected that food allergies are caused by the type II and type IV allergies in addition to the type I allergy. When an allergen invades the living body, the production of an antibody specific to the allergen is induced. An IgE antibody plays an important role specifically in the onset of the type I allergy. IgE produced by B cells binds to a high-affinity IgE receptor occurring on cell membrane of mast cells and basophils. When the allergen invades again to form crosslinkage of IgE on the mast cells and basophils, a mediator such as histamine is released, which results in the onset of the allergy.

Thus, the inventor has focused specifically on the IgE antibody and the IgE receptor participating in the allergy onset and examined effects of chrysin on the production and expression thereof.

IgE, which is an immunoglobulin, is a biomolecule serving as the key factor in immediate allergy onset. In the serum of a healthy human subject, the IgE production level (300 ng/ml) is much lower than the production level of IgG (15 mg/ml) which is also an immunoglobulin. In an allergic patient, however, overproduction of IgE is observed and it is considered that the IgE overproduction is one of the factors causing the onset of various allergic diseases typified by pollinosis and food allergies. IgE produced by the allergen-sensitization binds to the high-affinity IgE receptor occurring on the surface of mast cells and basophils. When the allergen invading again under these circumstances binds to the IgE, the IgE is crosslinked and the receptor aggregates. Then, calcium is mobilized into the cells and a cell membrane-mediated signaling mechanism comes into operation. As a result, the cells are activated and degranulated, thereby releasing pharmacologically active substances such as histamine, an eosinophil chemotactic factor and a neutrophil chemotactic factor. At the same time, the arachidonic acid cascade in the cell membrane is activated and lipid mediators such as leukotriene, prostaglandin and thromboxane and cytokines such as interleukins are produced and secreted, thereby inducing immediate allergy. Thus, inhibition of the IgE production, which plays a highly important role in the allergy onset, is very effective from the viewpoints of preventing and treating allergy. There are five kinds of antibodies, i.e., IgM, IgD, IgG, IgE and IgA depending on the differences in heavy chains. The heavy chain constant region is an important region for the achievement of biological activities (binding to a cell and fixation of a complement) of immunoglobulin. Immediately after the differentiation into mature B cells, the B cells produce IgM class immunoglobulin molecules. Then the B cells are activated into the cytokine-responsive state and recombine a gene in the heavy chain constant region of another class in response to stimulus of the cytokine without changing the sequence in the heavy chain variable region. This recombination phenomenon is called “class switch” whereby IgE antibody is produced for the first time. In the IgE class switch, a primary transcript, which comprises the I region located in the upstream of the IgE heavy chain constant region (Cε) to the constant region, is transcribed prior to the DNA recombination and a germline transcript (εGT) comprising the I region and Cε is produced via splicing. Next, the DNA sequence between the S region of the IgM heavy chain constant region (Cμ) and the S region of Cε is removed from chromosome and drawn toward just upstream of the variable region. Thus, the mature Cε transcription arises from the promoter in the variable region. It is estimated that the role of the expression of εGT, which is never translated into a protein, resides in opening the chromosome structure in the vicinity of the constant region targeted by the recombination by the transcription, thereby facilitating the recombination. The expression of εGT essentially required in the IgE production is induced by IL-4. This cytokine binds to each receptor expressed on the membrane surface of B cells and crosslinks it. As a result, JAK kinase bonded to the cytoplasm side of the receptor is mutually phosphorylated and thus activated. The activated JAK induces tyrosine phosphorylation of the receptor. Subsequently, a transcription factor STAT6 binds to the phosphorylated tyrosine residue. STAT6 associated with the receptor is phosphorylated by JAK. The phosphorylated STAT6 forms a homo dimer, migrates into the nucleus and binds to the germline Cε promoter region, thereby inducing εGT expression.

As described above, the inhibition of the εGT expression brings about fundamental suppression of IgE production. It is known that human mature B cell line DND39 expresses εGT in response to a stimulus by IL-4. Thus, DND39 cells were incubated in the presence of IL-4 and chrysin and the effect of chrysin on the εGT expression was examined.

### 2. Materials and Method:

### Chrysin:

Chrysin was purchased from Sigma Chemical Co. (St. Louis, Mo.). Chrysin was dissolved in dimethyl sulfoxide (DMSO) and diluted with ethanol to give a concentration of 10% DMSO.

Cells and Cell Incubation:

Human mature B cell line DND39 was subcultured in a RPMI 1640 medium (Nissui), to which 5% of FBS (Intergen) had been added, at 37° C. and 5% CO₂ vapor saturation and maintained therein. The RPMI 1640 medium employed comprised 100 U/mL of penicillin (Meiji Seika), 100 gm/mL of streptomycin (Meiji Seika), 12.5 mM of NaHCO₃ (Wako) and 10 mM of HEPES (Wako).

Detection of Germline IgE Heavy Chain Transcript (εGT)]:

The density of DND39 cells was adjusted to 2×10⁵ cells/mL and incubated in the presence of IL-4 (25 U/mL) and chrysin at each concentration. A liquid cell incubation medium comprising 1×10⁷ or fewer cells was collected in a 15 mL centrifuge tube made of polypropylene and centrifuged at 300×g. After discarding the supernatant, the residue was washed with PBS once. The cells, that had been centrifuged followed by the discarding of the supernatant, were preserved in ice until the subsequent operation to minimize digestion of RNA by RNase. After adding 1 mL of Trizol (Invitrogen, Carlsbad, Calif.), the cells were thoroughly suspended by pipetting until no mass was observed any more. The obtained suspension was transferred into a 1.5 mL tube and allowed to stand for 5 min. After adding 200 μL of chloroform (Wako Pure Chemical Co., ltd.), the mixture was vigorously stirred, allowed to stand at room temperature for 3 min and then centrifuged at 12,000×g for 15 min. The upper layer (450 μL) was transferred into another 1.5 mL tube and 500 μL of isopropanol (Wako Pure Chemical Co., ltd.) was added thereto. After vigorously stirring, the mixture was allowed to stand at room temperature for 10 minutes. After centrifuging at 12,000×g for 10 min, the supernatant was discarded by decantation. Next, 1 mL of 75% ethanol was added to the residue and the obtained mixture was vigorously stirred and centrifuged at 12,000×g for 5 min. After discarding the supernatant, the total RNA was collected, dissolved in 10 μL of DPEC water and diluted 100-fold. Then, the RNA concentration was measured using a spectrophotometer Ultrospec 3000 (Pharmacia Biotech, Piscatway, N.J.).

To a 600 μL tube, 10 μg of RNA, 0.5 μg of oligo dT₂₀ and 20 pmol of an εGT reverse primer (5′-gAC gCT gAAggT TTTgTT gTCg-3′) were added to give a total volume of 13.8 L. After maintaining at 70° C. for 10 min, the mixture was cooled for 10 min. 2 μL of 10 mM dNTP (Amersham), 4 μL of 5× buffer, 0.1 μL of Rnase inhibitor (TAKARA) and 0.1 μL of 25 kU MMLV-reverse transcriptase (Amersham) were added to the 600 μL tube as described above. The mixture was maintained at 37° C. for 1 hour and then boiled for 5 min to thereby inactivate the enzyme. The cDNA thus synthesized was preserved at −20° C. The above procedure was followed with the use of GAPDH (glyceraldehyde-3-phosphate dehydrogenase) as a control.

PCR was conducted by using 0.5 U Taq DNA polymerase (Fermentas) in 10 μL capacity.

The PCR mixture comprised the following reagents.

10×PCR buffer (1 μL)

25 mM MgCl (1 μL)

2.5 mM dNTPs (0.8 μL)

Taq DNA polymerase (0.1 μL)

20 μM sense primer (0.5 μL)

20 μM anti-sense primer (0.5 μL)

cDNA prepared above (1 μL)

dH₂O (5.1 μL)

The sequences of the primers employed in PCR were as follows.

εGT-F: 5′-AggCTC CAC TgCCCg gCA CagAAA T-3′

εGT-R: 5′-Acg gAg gTggCA TTg gAg ggA Atg T-3′

G3PDH-F: 5′-gCT CagACA CCA Tgg ggA Agg T-3′

G3PDH-R: 5′-gTg gTg Cag gAg gCA TTg CTgA-3′

Using a thermal cycler (Whtman Bometra GmbH, Germany), the initial denaturation was conducted at 94° C. for 2 min. Next, the cDNA was amplified in 20 cycles (for εGT) or 13 cycles (for GPDH) of amplification, each cycle consisting of denaturation at 94° C. for 1 min, annealing at 60° C. for 1 min and extension at 72° C. for 1 min. The amplified PCR product was electrophoresed on a 1.5% agarose gel (the same procedure was carried out for each concentration and the control in 1/10 amount), denatured with an alkali and transferred onto a Hybond-N+ membrane (Amersham) by capillary blotting. After the transfer, the membrane was pre-hybridized in a random buffer (composition: 5×SSC(SSC: 0.3 M sodium citrate, 3 M NaCl, pH 7.0), 3M NaCl, 333 mM C₆H₅O₇Na.2H₂, 0.1% SDS, 5% (w/v) dextran sulfate sodium salt) at 60° C. for 2 hours. Subsequently, 3 μL portions, per 5 mL of the same buffer, of εGT-P (1/40) and GAPDH-P having been preliminarily prepared by oligo labeling were added and incubation was carried out at 60° C. overnight. After the completion of the hybridization, the membrane was taken out and washed with washing liquors, each having been prepared by adding the following reagents and adjusted to a total volume of 100 mL by adding ultrapure water, in the order as shown below.

1st washing: 20×SSC (5 mL)+10% SDS (1 mL): 30 min: 60° C.
2nd washing: 20×SSC (2 mL)+10% SDS (1 mL): 30 min: 60° C.
3rd washing: 20×SSC (1 mL)+10% SDS (1 mL): 60 min: 60° C.

After the completion of the washing, the membrane was rinsed with buffer A (composition: 100 mM Tris-HCl pH 9.5, 300 mM NaCl). Then, blocking was conducted by adding 3 mL of a blocking reagent for blocking antibody (Amersham #1059304) diluted 10-fold with 1× buffer A thereto and shaking the mixture at room temperature for 2 hours. Next, 5 mL of alkali phosphatase-labeled antifluorescein antibody diluted 5000-fold with 0.5% BSA-buffer A was added and the membrane was reacted with the antibody by shaking at room temperature for 1 hour. After the completion of the reaction with the antibody, the membrane was washed thrice at room temperature with 0.67% Tween-buffer A each for 20 min and finally lightly rinsed with buffer A. The membrane was sandwiched between plastic sheets and 200 μL per membrane of CDP-Star was evenly sprayed thereon. After allowing to stand at room temperature for 1 min, εGT was detected with an image analyzer.

Detection of Phosphorylated STAT6:

The density of DND39 cells was adjusted to 2×10⁶ cells/mL and IL-4 (100 U/mL) and chrysin at each concentration were added thereto. Then, the cells were lysed by treating for 30 min. The cell lysate was subjected to immunoprecipitation using anti-STAT6 antibody. The immunoprecipitate was subjected to 8% SDS-PAGE and blotted onto a nitrocellulose membrane. Then phosphorylated STAT6 was detected using anti-tyrosine phosphorylation antibody (PY20).

### 3. Results:

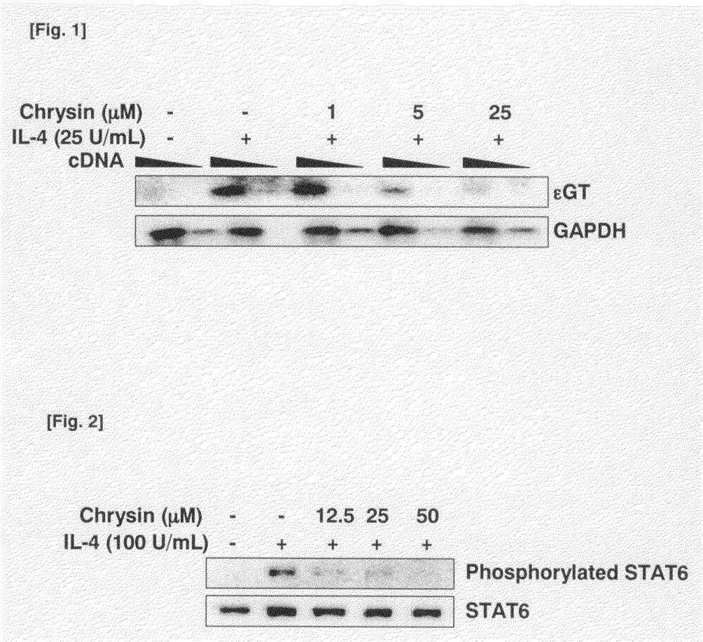

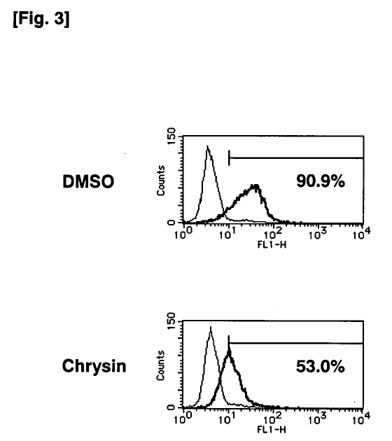

FIGS. 1 and 2 show the results. Chrysin concentration-dependently suppressed εGT expression. It also suppressed phosphorylation of STAT6. Thus, it can be understood that chrysin inhibits εGT expression at the STAT6 phosphorylation level and thus fundamentally suppresses IgE production.

### Example 2

### Suppression of Human High Affinity IgE Receptor (FcεRI) Expression

### 1. Object:

When an allergen invades the living body, the production of an antibody specific to the allergen is induced. An IgE antibody plays an important role specifically in the onset of immediate allergy. IgE produced by B cells binds to a high-affinity IgE receptor occurring on cell membrane of mast cells and basophils. When the allergen invades again to form crosslinkage of IgE on the mast cells, a mediator such as histamine or leukotriene is released, which results in the onset of the allergy.

FcεRI, which is a molecule serving as the key factor in the onset stage of an allergic reaction wherein IgE participates, drives a signal activating the mast cells and basophils expressing FcεRI via an antigen/antibody complex. The activation of FcεRI is induced by the aggregation of FcεRI. That is, IgE binding to FcεRI is crosslinked by a polyvalent antigen or the like and the signal transduction mechanism starts up due to the aggregation of FcεRI. To induce the aggregation of FcεRI, multiple FcεRI molecules should be present at such intervals as allowing the crosslinkage by the polyvalent antigen. Namely, the sensitivity of cells to the allergen is determined depending on the number of FcεRI molecules. In practice, FcεRI is hyperexpressed in, for example, mast cells in the nasal mucosa of a patient suffering from allergic rhinitis.

Suppression of FcεRI expression results in suppression of degranulation induced by the IgE/FcεRI complex. Thus, it is expected that the subsequent allergic symptoms, etc. can be alleviated thereby. KU812 cells known as a human basophil cell line produce histamine, which is an inflammatory substance, and express the high-affinity IgE receptor FcεRI on the cell membrane. Thus, the effect of chrysin on FcεRI expression was examined by using these KU812 cells.

### 2. Materials and Method:

### Cells and Cell Incubation:

As a basal medium for incubating cells, use was made of RPMI 1640 (Nissui, Japan), to which 100 U/mL of penicillin (Meiji Pharmaceutical Company, Tokyo, Japan), 100 mg/mL of streptomycin (Meiji Pharmaceutical Company, Tokyo, Japan), 12.5 mM of MaHCO₃ (Wako Pure Chemical Industries, Osaka, Japan) and 10 mM of HEPES Wako Pure Chemical Industries) had been added. Fetal bovine serum (FBS) purchased form Bio Source International was added to the basal medium to give a concentration of 5 to 10%. Human basophil-like cell line KU812 employed in this experiment was assigned by Japan Collection of Research Bioresources (JCRB) and subcultured and maintained in RPMI 1640 medium comprising 5 to 10% of FBS at 37° C. in 5% CO₂ gas under humidification.

Chrysin:

Chrysin (Sigma) was dissolved in dimethyl sulfoxide (DMSO) and diluted with ethanol to give a concentration of 10% DMSO

Analysis of FcεRI Expression Amount on Cell Surface:

KU812 cells were incubated in RPMI 1640 medium, to which chrysin dissolved in DMSO had been added, for 0 to 24 hours. A system wherein the cells were incubated in the medium comprising DMSO alone was used as a control. After incubating, the cells were collected and incubated in 5% FBS-PBS, to which 10 μg/mL of antihuman FcεRI α chain mouse monoclonal antibody CRA-1 (Kyokuto Pharmaceutical) had been added, at 4° C. for 40 to 60 min. As a negative control, 10 μg/mL of mouse control IgG2b antibody (DAKO) was used. Then, the cells were washed with ice-cooled PBS once and incubated in 5% FBS-PBS, to which 10 μg/mL of antimouse IgG2b FITC-labeled antibody (Protos Immunoresearch) had been added, at 4° C. for 40 to 60 min. After incubating, the cells were washed with PBS and then resuspended in PBS. The cell suspension thus obtained was analyzed by flow cytometry. In the flow cytometry analysis, FACS Calibur (Becton Sickinson, Sunnyvale, Calif.) was employed. The secondary antibody reaction and the subsequent procedures were conducted in the dark and the cells were preserved on ice while conducting the analysis.

Examination on Expression Levels of Proteins Constituting FcεRI:

KU812 cells (1×10⁶ cells/mL) were incubated under serum-free conditions in RPMI 1640 medium, to which chrysin had been added, for 6 hours. After incubating, the cells were washed with PBS once and then lysed by shaking together with Lysis buffer (composition: 50 mM Tris-HCl pH 7.5, 1% Triton X-100, 150 mM NACl, 1 mM EDTA, 50 mM NaF, 30 mM Na₄P₂O₇, 1 mM PMSF, 2 μg/mL aprotinin, 1 mM pervandate) at 4° C. for 30 min. Subsequently, the mixture was centrifuged at 12,000×g for 10 min at 4° C. to precipitate insoluble matters. The supernatant was collected and proteins were quantified. The samples were diluted with Lysis buffer so that all of them had the same protein concentration, thereby giving lysate samples.

Protein A Sepharose beads (Amersham Pharmacia Biotech) were shaken in Lysis buffer (composition: 50 mM Tris-HCl pH 7.5, 150 mM NACl, 1% Triton X-100, 1 mM EDTA, 50 mM NaF, 30 mM Na₄P₂O₇, 1 mM PMSF, 2 μg/mL aprotinin), to which CRA-1 antibody had been added to give a final concentration of 1 μg/mL, for 2 to 4 hours at 4° C. so as to allow the beads to adsorb CRA-1 antibody. After collecting, the beads were washed with Lysis buffer thrice and the cell lysate sample was added thereto. Then, immunoprecipitation was conducted by shaking the mixture at 4° C. overnight. After the immunoprecipitation, the Protein A Sepharose beads were collected and washed with Lysis buffer and PBS three time each. After washing, 20 to 30 μL of a sodium dodecyl sulfate-polyacrylamide gel electrophoresis (SDS-PAGE) sample buffer (0.057 M Tris-HCl pH 6.8, 1.8% (w/v) SDS, 0.65 M β-mercaptoethanol, 9.1% glycerol, 0.02% bromophenol blue) was added to the Protein A Sepharose beads and the mixture was stirred in a vortex for 10 min. The immunoprecipitate thus separated was thermally denatured at 95° C. for 5 min. The sample employed for detecting FcεRI γ chain was prepared in such a manner as to give a ratio of the cell lysate to the sodium dodecyl sulfate-polyacrylamide gel electrophoresis (SDS-PAGE) sample buffer of 1:1 and thermally denatured at 95° C. for 5 min. Then, SDS-PAGE was conducted by applying the immunoprecipitate sample to a 10% polyacrylamide gel in the case of FcεRI α chain, or applying the cell lysate sample to a 15% polyacrylamide gel in the case of FcεRI γ chain.

After conducting SDS-PAGE, each gel was subjected to electroblotting at 100V (α chain: 60 min, γ chain: 30 min) and the protein was transferred onto a nitrocellulose membrane. This membrane was shaken in 0.05% Tween 20-TBS (TTBS) to which skim milk had been added to give a final concentration of 2% and the blocking reaction was performed at room temperature for 30 to 60 min. After washing with TTBS, 0.050 μg/mL of CRA-1 and anti-FcεRI γ-chain antibody (Upstate biotechnology) were added and the mixture was reacted at 4° C. overnight. After washing with TTBS, HRP-labeled antimouse IgG antibody and HRP-labeled antirat IgG antibody diluted 1000-fold were added thereto and the mixture was reacted at room temperature for 1 hour. These antibodies were diluted with 2% skim milk-TTBS employed in the blocking. After washing with TTBS, a color development reaction was conducted using a high sensitivity ECL (ECL Advance Western Blotting Detection Kit) and detection was performed by exposing a Polaroid film to light. As a control, β-actin was employed.

Examination of mRNA Transcription Level:

KU812 cells (1×10⁶ cells/mL) were incubated under serum-free conditions in RPMI 1640 medium to which chrysin had been added. After incubating, the cells were washed with PBS once and then completely lysed by adding 1 mL of Trizol (Invitrogen) per 1×10⁷ cells and quickly suspending. After allowing to stand at room temperature for 5 min, 0.2 mL of chloroform was added and the mixture was vigorously stirred and overturned. After allowing to stand at room temperature for 3 min, it was centrifuged at 12,000×g at 4° C. for 15 min. 450 μL of the supernatant was collected and 0.5 mL of 2-propanol was added. Then the mixture was vigorously stirred and overturned. After allowing to stand at room temperature for 10 min and then centrifuged at 12,000×g for 10 min at 4° C., the supernatant was removed and the precipitate was rinsed with 1 mL of 75% ethanol. After centrifuging at 12,000×g for 5 min at 4° C., the supernatant was removed as far as possible and the RNA precipitate was dissolved in to 10 20 μL of DEPC water. The concentration of the total RNA diluted 50-fold with water was measured using a wavelength of 260 nm. The samples were diluted with 0.1% diethyl pyrocarbonate (DEPC) water (Sigma) so that all of the samples had a total RNA concentration of 10 μg.

Complementary DNA (cDNA) was synthesized by adding 1.0 μL of a (dT)₂₀ primer per 10 μg of the total RNA, incubating at 70° C. for 10 min, then immediately cooling with ice and conducting an annealing reaction. Next, 2.0 μL of 10 mM dNTP, 0.1 μL of RNase inhibitor (Takara), 0.1 μL of MMLV-reverse transcriptase (Amersham Pharmacia Biotech) and 4.0 μL of 5× buffer accompanying the MMLV-reverse transcriptase were added thereto and the total volume was adjusted to 20 μL with DEPC water. The obtained mixture was incubated at 37° C. for 1 hour to thereby synthesize cDNA. Using the thus synthesized cDNA as a template, PCR was carried out with the use of primers specific to the FcεRI α- and γ-chains and glyceraldehyde-3-phosphate dehydrogenase (G3PDH). The sequences of the sense and antisense primers were as follows.

Human FcεRI α chain sense primer:
5′ -CTTAGGATGTGGGTTCAGAAGT- 3′
Antisense primer:
5′ -GACAGTGGAGAATACAAATGTCA- 3′
Human FcεRI γ chain sense primer:
5′ -TAGGGCCAGCTGGTGTTAATGGCA- 3′
Antisense primer:
5′ -GATGATTCCAGCAGTGGTCTTGCT- 3′
Human G3PDH sense primer:
5′ -GCTCAGACACCATGGGGAAGGT- 3′
Antisense primer:
5′ -GTGGTGCAGGAGGCATTGCTGA- 3′

The PCR mixture was prepared by mixing 1 μL of the cDNA, 0.8 μL of 2 mM dNTP, 0.6 μL of MgCl₂, 0.5 μL of 0.20 μM sense primer, 0.5 μL of 20 μM antisense primer, 0.1 μL of Taq DNA polymerase (Fermentas) and 1 μL of 10× Taq buffer accompanying Taq DNA polymerase and adjusting the total volume to 10 μL with distilled water. This liquid mixture was subjected to PCR. The PCR was conducted by using Gene Amp PCR System 2400 (Parkin-Elmer). In the PCR, the initial denaturation was conducted at 94° C. for 2 min. Next, amplification was conducted in 13 to 15 cycles (each cycle consisting of 1 min at 95° C., 1 min at 60° C. and 1 min at 72° C. The PCR product was electrophoresed on a 1% agarose gel and the amplified DNA was detected by Southern blotting.

After the completion of the electrophoresis, the gel was shaken in a 0.5N NaOH solution comprising 9% of NaCl at room temperature for 1 hour. Next, the DNA was blotted on a Hybond-N⁺ membrane by capillary blotting over 5 hours or longer with the use of 0.4 N NaOH. After blotting, the membrane was washed with 2× saline sodium citrate (SSC; 0.3 M sodium citrate, 3 M NaCl, pH 7.0). Probes of FcεRI α- and γ-chains and G3PDH were prepared by the random labeling method. First, the target DNAs having been amplified by RT-PCR were subcloned and the base sequences were analyzed with a DNA sequencer. Thus, it was confirmed that the subcloned DNAs were respectively specific to FcεRI α- and γ-chains and G3PDH. Using these DNAs as templates, PCR was conducted with the use of primers respectively specific thereto. Next, the amplification products were labeled in accordance with the protocol attached to Gene Images random labeling module. The membrane having each PCR product blotted thereon was dipped in a prehybridization buffer attached to Gene Images random labeling module and shaken at 60° C. for 60 min. Then, the buffer was replaced by a probe solution diluted 1000-fold with the prehybridization buffer and the membrane was incubated at 60° C. overnight. Next, the membrane was washed by shaking successively in 0.1 SDS-1×SSC for 20 min at 60° C., 0.1% SDS-0.4×SSC for 20 min at 60° C. and 0.1% SDS-0.2×SSC for 20 min at 60° C. After washing the membrane with buffer A attached to a CDP-Star detection module kit (Amersham Pharmacia Biotech), the membrane was shaken in a blocking solution attached to the kit for 1 hour at room temperature. Alkali phosphatase-labeled antifluorescein antibody attached to the kit was diluted 5000-fold with buffer A comprising 0.5% of bovine serum albumin (Roche Diagnistic GmbH, Mannheim, Germany). The membrane was dipped in this antibody solution and shaken for 60 min at room temperature. After washing with buffer A comprising 0.3% of Tween 20 thrice each for 15 min, the membrane was further washed with buffer A once. Then, color development was conducted by dropping CDP-Star that was a substrate of the alkali phosphatase attached to the kit onto the membrane and then detection was performed by exposing a Polaroid film to light.

### 3. Results:

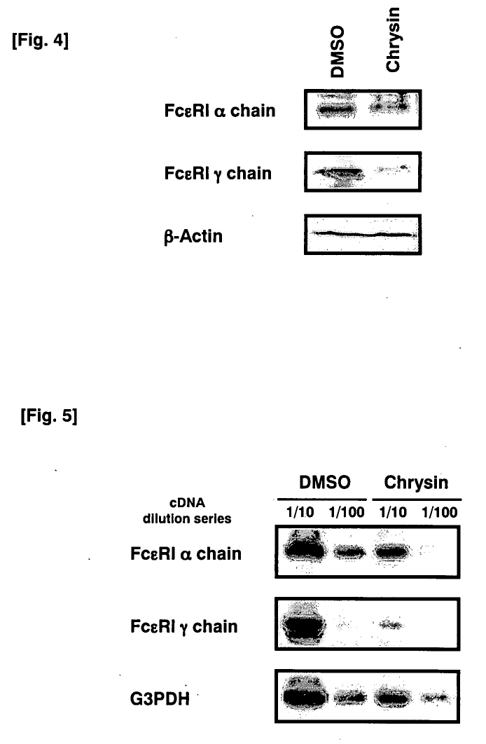

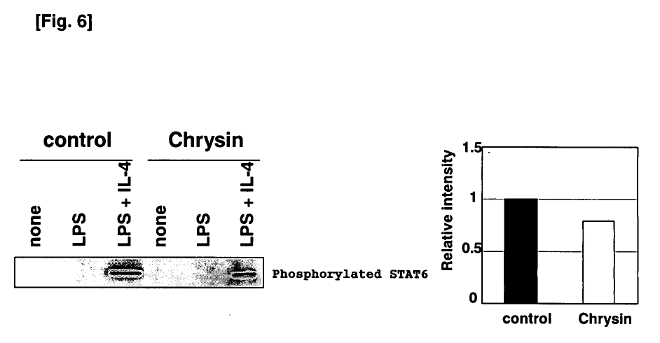

FIGS. 3 to 5 show the results. Chrysin suppressed the expression of FcεRI at the mRNA level. It can be understood that chrysin suppresses the expression of FcεRI and, in its turn, suppresses degranulation induced by the IgE/FcεRI complex and alleviates the subsequent allergic inflammation and so on.

### Example 3

### Chrysin Intake Test

### Samples Administered:

200 μL portions of 0.4 mg/mL chrysin and 200 μL portions of 1% DMSO in 1% EtOH were administered respectively to groups (n=8) in accordance with the following administration schedule.

Administration Schedule:

To male C57BL/6N mice aged 5 weeks that had been preliminarily fed (MF feed) for 1 week, the samples were administered once every 2 days for 2 weeks (7 times in total; total dose: 0.56 mg (28 mg/kg) per animal).

### Examination on IL-4-Response:

After sacrificing, spleen lymphocytes were collected from the spleen of each mouse, adjusted to a density of 2×10⁷ cells/mL and stimulated with 25 μg/mL of lipopolysaccharide (LPS) or LPS+IL-4 (500 U/mL) for 30 min. Then, the cells were lysed and phosphorylated STAT6 was detected by immunoprecipitation with the use of anti-STAT6 antibody and immunoblotting to thereby examine the response to IL-4.

Results:

FIG. 6 shows the results. In the chrysin-administered system, the phosphorylation of STAT6 by the stimulus with LPS+IL-4 was suppressed compared with the control system.

### Example 4

### Effect of Chrysin on the Expression of Human Peripheral Blood Basophil FcεRI mRNA

### 1. Method:

Venous blood was collected from the forearm elbow of a donor. Then, peripheral blood lymphocytes were separated and purified using Lymphocyte Separation Medium (ICN Biomedical Inc.) and peripheral basophils were separated and purified by the density gradient centrifugation method using Percoll (Amersham Biosciences). The basophils (1×10⁶ cells/mL) were incubated in 5% FCS-RPMI 1640 medium, to which 25 M of chrysin or a solvent dimethylsulfoxide (DMSO) had been added, for 24 hours. RNA was extracted and the mRNA expression levels of FcεRI α- and γ-chains were examined by the RT-PCR method as in Example 1.

### 2. Results:

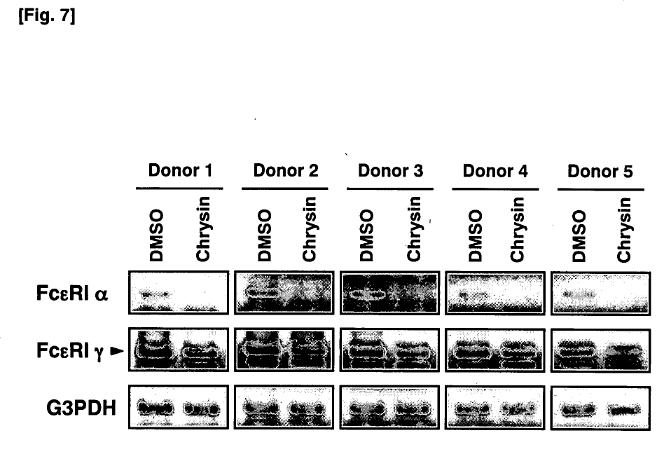

FIG. 7 shows the results. Chrysin lowered the mRNA expression levels of FcεRI α- and γ-chains in every donor. In particular, it strongly lowered the FcεRI α-chain expression level. Thus, it can be understood that chrysin strongly suppresses the expression of the high-affinity IgE receptor FcεRI in human peripheral blood cells too.

### Example 5

### Effect of Chrysin on Histamine Release from Human Basophil

### 1. Method:

Human basophil cell line KU812 cells (5×10⁵ cells/mL) were incubated in Tyrode buffer (137 mM NaCl, 2.7 mM KCl, 1.8 mM CaCl₂, 1.1 mM MgCl₂, 1.19 mM NaHCO₃, 0.4 mM NaH₂PO₄, pH 7.4) comprising 25 μM of chrysin and 1 mM of CaCl₂ for 20 min at 37° C. After adding 5 μM of A23187 (Sigma Chemical Co.), the incubation was continued for an additional 20 min at 37° C. After incubating, the reaction was ceased by allowing the reaction mixture to stand in ice for 5 min and 1 mL of the supernatant was collected. In a sample for quantifying histamine cumulated in cells, the cells were centrifuged at 300×g for 5 min, ultrasonically disrupted for about 1 min and then centrifuged at 12,000×g for 5 min and 1 mL of the supernatant was collected. To perform fluorescence measurement under alkaline conditions, a complex of histamine and o-phthaldehyde (Wako) was formed and partly extracted. 1 mL of the sample, 1 mL of Tyrode buffer, 0.75 to 0.78 g of NaCl and 0.5 mL of 5 N NaOH were mixed and stirred. Further, 5 mL of a n-butanol:chloroform mixture (3:2 v/v) was added thereto and the resultant mixture was stirred in a vortex mixer and then centrifuged at 300×g for 5 min. 4 mL of the upper layer was collected and 1.5 mL of 0.1 N HCl and 2 mL of n-heptane were added thereto. The resultant mixture was stirred in a vortex mixer and then centrifuged at 300×g for 5 min. 1 mL of the lower layer was added to 0.15 mL of 1 N NaOH and 0.1 mL of 0.2% OPA (prepared by dissolving in methanol) was added thereto followed by the initiation of the fluorescent reaction. After reacting for 5 min at room temperature, the reaction was ceased by adding 0.14 mL of 0.5 N H₂SO₄. The fluorescent intensity was measured using a fluorophotometer RF-1500 at an excitation wavelength of 360 nm and a fluorescent wavelength of 450 nm.

### 2. Results:

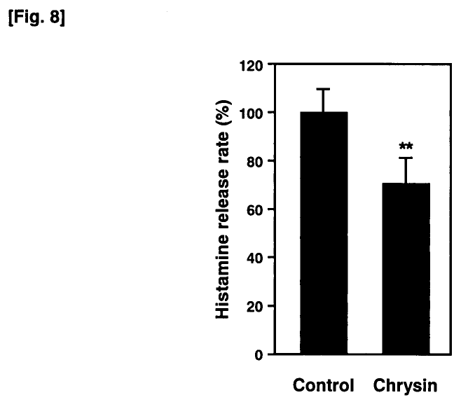

FIG. 8 shows the results.

Chrysin significantly lowered histamine release induced by A23187. Thus, it can be understood that chrysin inhibits the release of histamine that is an inflammatory substance induced by an antigen-stimulus.

### Example 6

### Effect of Chrysin Intake on Blood Antibody Level of Mouse Antigen-Sensitized with Ovalbumin (OVA)

### 1. Method:

Male BALB/c mice aged 8 weeks were divided into an AIN-93M-fed group (a control group) and a chrysin-comprising AIN-93M-fed group. After feeding with the individual feeds for 3 weeks, both groups were fed with the AIN-93M feed for 2 weeks. Each feed was given at a ratio of 5 g/day/animal while the animals were allowed to take water ad libitum.

OVA-sensitization was conducted by injecting 100 μg of OVA/aluminum hydroxide gel into the peritoneal cavity of each mouse 1 and 2 weeks after the initiation of the feed, i.e., twice in total.

On day 35, blood samples were collected and the total IgM, IgG and IgE levels in the serum were measured by ELISA.

Reagents employed in ELISA for quantifying the antibody were as follows. In blocking, use was made of PBS comprising 1% of bovine serum albumin (Roche) (BSA-PBS). As solid phase antibodies, use was made of F(ab′)2 goat anti-mouse IgG antibody (Zymed Laboratories, Inc.), F(ab′)2 rabbit anti-mouse IgM antibody (Zymed Laboratories, Inc.) and rabbit anti-mouse IgA (Zymed Laboratories, Inc.). As secondary antibodies, use was made of HRP-labeled F(ab′)2 goat anti-mouse IgG(H(L) antibody (Zymed Laboratories, Inc.), HRP-labeled rabbit anti-mouse IgM antibody (Zymed Laboratories, Inc.) and HRP-labeled goat anti-mouse IgA antibody (Zymed Laboratories, Inc.). 2,2′-Anizo-bis(3-ethylbenzothiazoline-6-sulfonic acid) diammonium salt (ABTS) employed as a color development substrate was purchased from Wako Pure Chemical Industries, while oxalic acid was purchased from Nacalai tesque (Kyoto, Japan).

A solid phase antibody solution (diluted 1000-fold with a 50 mM carbonate buffer) was pipetted into an immunoplate at a ratio of 100 μL/well and allowed to stand overnight at 4° C. After washing thrice with PBS comprising 0.1% of polyoxyethylene sorbitan monolaurate (Nacalai tesque) (TPBS), 300 μL/well of BSA-PBS was pipetted and incubation was conducted for 2 hours at 37° C. After washing thrice with TPBS, a sample and a standard solution, which were optionally diluted with BSA-PBS, were added at a ratio of 50 μL/well and allowed to stand overnight at 4° C. After washing thrice with TPBS, 100 μL/well of a secondary antibody solution (diluted 2000-fold with BSA-PBS) was pipetted and incubation was conducted for 1 hour at 37° C. After washing thrice with TPBS, 100 μL/well of a color development substrate solution was pipetted and color development was carried out. Then, 100 μL/well of oxalic acid was pipetted to cease the reaction and the absorbance was measured at 415 to 490 nm. As the color development substrate solution, a mixture of a 0.006% H₂O₂— comprising 0.2 M citrate buffer (pH 4.0), deionized water and a 10.94 mM ABTS solution (10:9:1) was employed.

In the case of IgE, the procedure indicated in the protocol attached to a mouse IgE assay kit (Morinaga Biochemistry Lab.) was followed. Absorbance was measured using Immuno Mini NJ-2300 (Nippon Intermed).

### 2. Results:

The following table shows the results.

[Table 1]

TABLE 1
Effect of chrysin intake on blood antibody level
of mouse antigen-sensitized with ovalbumin (OVA)

| Ig | Control | Chrysin |
|---|---|---|
| IgM (mg/mL) | 5.2 ± 0.2 | 5.4 ± 0.2 |
| IgG (mg/mL) | 27.7 ± 2.4 | 23.5 ± 3.0 |
| IgA (mg/mL) | 0.6 ± 0.1 | 0.6 ± 0.0 |
| IgE (mg/mL) | 87.5 ± 6.9 | 68.2 ± 4.2* |

At the point of terminating the intake, no effect of chrysin intake was observed in the blood IgM, IgG and IgA levels. In contrast thereto, the blood IgE level was significantly lowered compared with the control group, which indicates that chrysin intake suppresses an increase in the blood IgE level caused by the OVA sensitization.

### Example 7

### Effect of Chrysin Intake on Blood Cytokine Levels

### 1. Method:

Blood was collected as in Example 6 and 32 kinds of cytokines (6 Ckine, CTACK, Eotaxin, G-CSF, GM-CSF, IL-2, IL-3, Il-4, IL-5, Il-6, IL-9, IL-10, IL-12, IL-12p70, IL-13, IL-17, IFN-γ, KC, Leptin, MCP-1, MCP-5, MIP-1α, MIP-2, MIP-3β, RANTES, SCF, sTNFRI, TARC, TIMP-1, TNF-α, Tpo and VEGF) were detected using Ray Bio™ Mouse Cytokine Array (Ray Biotech, Inc.).

The abbreviations have the following meanings: CTACK: cutaneous T-cell-attracting chemokine; G-CSF: granulocyte colony-stimulating factor; GM-CSF: granulocyte-macrophage colony-stimulating factor; IFN: interferon; KC: CXC ligand 1; MCP: monocyte chemoattractant protein; MIP: macrophage inflammatory protein; RANTES: regulated upon activation normal T cell expressed and secreted; SCF: stem cell factor; sTNFR: soluble tumor necrosis factor receptor; TARC: thymus and activation-regulated chemokine; TIMP-1: tissue inhibitor of metalloprotease; TNF: tumor necrosis factor; Tpo: thrombopoietin; and VEGF: vascular endothelial growth factor.

### 2. Results:

The following table shows the results wherein each numerical value indicates a relative value calculated by referring the value of the non-intake group as to 1.

[Table 2]

TABLE 2
Effect of chrysin intake on blood cytokine levels
of mouse antigen-sensitized with ovalbumin (OVA)

| Cytokine | Chrysin |
|---|---|
| G-CSF | 2.43 |
| GM-CSF | 1.68 |
| IL-4 | 0 |
| Leptin | 1.25 |
| MCP-1 | 0.49 |
| TNF-α | 2.17 |

Among these 32 kinds of cytokines, 26 ones showed no change in the cytokine levels. In contrast thereto, the IL-4 level was remarkably lowered by taking chrysin (lower than the detection limit). On the other hand, the blood levels of G-CSF, GM-CSF, leptin and TNF-α showed a tendency toward increase.

### Example 8

### Effect of Coexistence of Chrysin and Apigenin on Expression of High-Affinity IgE Receptor (FcεRI) on Cell Surface

### 1. Method:

KU812 cells (5×10⁵ cells/mL) was incubated in 5% bovine fetal serum comprising RPMI 1640 medium, to which chrysin (0, 1, 5 and 10 μM) and apigenin (purchased from Aldrich Chem. Co., St. Louis, Mo.; 0, 1, 5 and 10 μM) had been added, for 24 hours. After incubating, the FcεRI expression level on the cell surface was measured by flow cytometry (experimental procedure: see [0047]).

### 2. Results:

The following table shows the results.

[Table 3]

TABLE 3
Effect of coexistence of chrysin and apigenin on expression
of high-affinity IgE receptor (FcεRI) on cell surface

| Chrysin/apigenin | FcεRI expression |
|---|---|
| combination | suppressive rate (%) |
| | 0.0 |
| Apigenin 1 μM | 5.0 |
| Apigenin 5 μM | 17.1 |
| Apigenin 10 μM | 36.1 |
| Chrysin 1 μM | 0.0 |
| Chrysin 1 μM + Apigenin 1 μM | 5.4 |
| Chrysin 1 μM + Apigenin 5 μM | 26.0 |
| Chrysin 1 μM + Apigenin 10 μM | 40.8 |
| Chrysin 5 μM | 11.5 |
| Chrysin 5 μM + Apigenin 1 μM | 13.9 |
| Chrysin 5 μM + Apigenin 5 μM | 31.4 |
| Chrysin 5 μM + Apigenin 10 μM | 51.4 |
| Chrysin 10 μM | 19.8 |
| Chrysin 10 μM + Apigenin 1 μM | 22.2 |
| Chrysin 10 μM + Apigenin 5 μM | 39.8 |
| Chrysin 10 μM + Apigenin 10 μM | 58.0 |

Although 1 μM of chrysin did not suppress the FcεRI expression in KU812 cells, it potentiated the effect of apigenin of suppressing the FcεRI expression. In particular, a synergistic effect of suppressing the FcεRI expression (suppressive rate: 26%) was observed in the case (1 μM of chrysin+5 μM of apigenin) wherein coincubation was conducted using 1 μM of chrysin (suppressive rate: 0%) with 5 μM of apigenin (suppressive rate: 17.1%).

1. An allergy suppressive agent comprising chrysin.

2. A histamine release inhibitory agent comprising chrysin.

3. A composition comprising chrysin for treating a disease or condition relating to the production of IgE.

4. A composition comprising chrysin for treating a disease or condition relating to a high-affinity IgE receptor.

5. A composition comprising chrysin for treating a disease or condition relating to the overproduction of IL-4.

6. A pharmaceutical or food composition which comprises 1.68 g or more of chrysin in a daily oral dose or intake.

7. The allergy suppressive agent according to claim 1, the histamine release inhibitory agent according to claim 2 or a composition according to any one of claims 3 to 5, which further comprises apigenin.

8. A pharmaceutical, food or cosmetic composition which comprises chrysin and apigenin.

9. Use of chrysin in the manufacture of an allergy suppressive agent, a histamine release inhibitory agent, a composition for treating a disease or condition relating to the production of IgE, a composition for treating a disease or condition relating to a high-affinity IgE receptor or a composition for treating a disease or condition relating to the overproduction of IL-4.

10. A method for treating a disease or condition relating to allergy, a disease or condition relating to histamine release, a disease or condition relating to the production of IgE, a disease or condition relating to a high-affinity IgE receptor or a disease or condition relating to the overproduction of IL-4, which comprises administering a therapeutically effective amount of chrysin to a mammal with a need for such a treatment.


SEQUENCE LISTING (10 sequences)

SEQ ID NO: 1 - DNA, 25 bp, Artificial Sequence
  epsilon GT-F, Synthetic PCR primer
aggctccactgcccggcacagaaat

SEQ ID NO: 2 - DNA, 25 bp, Artificial Sequence
  epsilon GT-R, Synthetic PCR primer
acggaggtggcattggagggaatgt

SEQ ID NO: 3 - DNA, 22 bp, Artificial Sequence
  G3PDH-F, Synthetic PCR primer
gctcagacaccatggggaaggt

SEQ ID NO: 4 - DNA, 22 bp, Artificial Sequence
  G3PDH-R, Synthetic PCR primer
gtggtgcaggaggcattgctga

SEQ ID NO: 5 - DNA, 22 bp, Artificial Sequence
  human Fc epsilon RI alpha chain, sense primer
cttaggatgtgggttcagaagt

SEQ ID NO: 6 - DNA, 23 bp, Artificial Sequence
  human Fc epsilon RI alpha chain, antisense primer
gacagtggagaatacaaatgtca

SEQ ID NO: 7 - DNA, 24 bp, Artificial Sequence
  human Fc epsilon RI gamma chain, sense primer
tagggccagctggtgttaatggca

SEQ ID NO: 8 - DNA, 24 bp, Artificial Sequence
  human Fc epsilon RI gamma chain, antisense primer
gatgattccagcagtggtcttgct

SEQ ID NO: 9 - DNA, 22 bp, Artificial Sequence
  human G3PDH, sense primer
gctcagacaccatggggaaggt

SEQ ID NO: 10 - DNA, 22 bp, Artificial Sequence
  human G3PDH, antisense primer
gtggtgcaggaggcattgctga